(19) United States
(12) Patent Application Publication
(10) Pub. No.: US 2023/0419421 A1
(43) Pub. Date: Dec. 28, 2023

(54) GENOMIC TECHNOLOGIES FOR AGRICULTURE PRODUCTION AND PERFORMANCE MANAGEMENT

(71) Applicant: AGRIC-BIOFORMATICS, INC., Oklahoma City, OK (US)

(72) Inventor: Sean AKADIRI, Oklahoma City, OK (US)

(21) Appl. No.: 18/463,132

(22) Filed: Sep. 7, 2023

Related U.S. Application Data
(63) Continuation of application No. 15/743,898, filed on Jan. 11, 2018, now abandoned.
(60) Provisional application No. 62/192,598, filed on Jul. 15, 2015.

(51) Int. Cl. G06Q 50/02; G06Q 10/0635; C12Q 1/6895; C12N 15/10; G06Q 10/0639; G06Q 30/018; G06Q 10/06; G01N 35/00

(52) U.S. Cl. CPC G06Q 50/02; G06Q 10/0635; C12Q 1/6895; C12N 15/1075; G01N 2035/00831; G06Q 30/018; G06Q 10/06; G01N 35/00732; G06Q 10/0639

(57) ABSTRACT

An agriculture management and analysis system for analysis, interpretation, and visualization of genetic data to improve production, performance, and management of agriculture, such as livestock.

### CROSS-REFERENCE TO RELATED APPLICATION

The present application claims priority under 35 U.S.C. § 119(e) to U.S. Provisional Patent Application Ser. No. 62/192,598, entitled “GENOMIC TECHNOLOGIES FOR AGRICULTURE PRODUCTION AND PERFORMANCE MANAGEMENT,” which was filed on Jul. 15, 2015, which is incorporated herein by reference in its entirety.

### TECHNICAL FIELD

The present disclosure relates to genomic technologies for agriculture production and performance management. More specifically, the present disclosure pertains to an agriculture management and analysis system for analysis, interpretation, and visualization of genetic data in order to improve production, performance, and management of agriculture, such as livestock and crops.

### BACKGROUND

Agriculture (e.g., growing crops, rearing livestock, etc.), despite its unstable and unpredictable nature, continues to be critically important to economies across the globe. Thanks to its natural resources and land conditions, the United States (U.S.) is one of the world's leading agricultural producers, particularly as it relates to crops and livestock. For example, the U.S. is the world's largest beef producer and second largest beef exporter. In fact, in 2012, total U.S. agricultural sales were $394.6 billion, with $182.2 billion of that total attributed to livestock sales revenue (United States Department of Agriculture (USDA) National Agricultural Statistics Service (NASS), 2012 Census of Agriculture). Most of these sales were from livestock or livestock products, such as cattle/calves (e.g., beef and dairy), poultry (e.g., eggs & chicken), and hogs (e.g., pork).

However, as the human population is estimated to be around 10 billion by the year 2050, there remain foreseeable challenges for livestock production systems to meet the increasing food demand, as well as global livestock production systems, which are expected to rise by 60% globally. With a projected 3 billion more people to feed worldwide, the global demand for foods, such as livestock and dairy products, will only continue to increase exponentially. To meet this demand, farmers will have to increase their agriculture production while encountering unpredictable environmental and economic challenges such as land losses, water scarcity, increasing pollution, political instability, limited bioenergy resources, and climate change.

Agriculture is the economic sector that is often hit hardest by climate change. For example, heat stress caused by higher temperatures due to global climate change can seriously impact crops and livestock, and over time can increase vulnerability to parasites and disease. Equally as damaging, drought can reduce the amount of quality forage available to livestock, and thus inhibit the health and well-being, and ultimately the economic value of livestock.

Accordingly, the U.S. Food and Drug Administration (FDA) has recently requested that animal health companies voluntarily alter their product labels to prevent farmers from using antibiotics in feed to promote enhanced growth of livestock. The use of antibiotics in livestock has been prevalent for over forty years. To date, many livestock producers feed low doses of antibiotics to animals to promote faster growth. The proposed U.S. FDA regulation requires ranchers to obtain a veterinarian prescription in order to use antibiotics to prevent disease in animals, and livestock producers will no longer be allowed to use antibiotics to enhance growth rate or improve feed efficiency. Accordingly, the new U.S. FDA regulation will likely create economic hardships for livestock producers who generally operate on thin margins.

The proposed government restriction on antibiotic usage in livestock, along with the projected increase in global food demand, makes the current decision-making of agriculture producers even more costly and critical. Traditionally, livestock ranchers have been accustomed to making farming and agriculture management decisions based on long-term experience, with the help of some standard operating procedures (SOPs). As such, genomics tools have gradually been adopted over the past decade by large agriculture companies and operations.

Companies such as Zoetis, Cargill, and Alltech have been involved with helping large producers to be more efficient when it comes to livestock production. However, the cost of such genetic services designed for large-scale livestock producers is typically beyond the reach of the majority of farmers. In addition, the methods of delivery, analysis, and interpretation of an individual producer's livestock genetic data can be very complex, time-consuming, and difficult to decode or decipher, especially for small farmers who may not be as technologically savvy.

In fact, such farmers do not typically have the resources available to evaluate decisions using such high-level technology. Instead, small producers are tied to a basic set of practices with limited windows of error to make adjustments in response to the market, weather, cost fluctuation, etc. For example, a major challenge for small producers is determining how to evaluate the quality of live animals by depending on commercial valuation data that is often inaccurate or difficult to understand. Likewise, a small farmer making informed decisions based on high-level genetic data may be unlikely.

The present disclosure is directed to address this problem and particularly relates to a software system or tool to help a user analyze, interpret, and visualize genetic data collected from animals to help with critical decision-making, and provides suggestions and recommendations regarding the same. In particular, this disclosure relates to a platform technology that provides genetic information comprising genetic profiles and breeding, nutrition, lineage tracing, and valuation of crops and animals. Ultimately, the technology of the present disclosure helps to promote optimal animal nutrition and management of agricultural performance and profitability.

### SUMMARY OF THE INVENTION

The present disclosure is directed to a method for managing agricultural products in an agricultural farm. The method comprises receiving, by a remote compute and store server, registration details from a user, wherein the registration details define one or more characteristics of an agricultural product. The method also comprises receiving, by a remote compute and store server, genetic data from a user, wherein the genetic data defines one or more gene markers of the agricultural product to be analyzed.

In addition, the method comprises analyzing the genetic data. The method also comprises generating a genetic profile of the agricultural product based on the genetic data. Further, the method comprises generating, by the remote compute and store server, a genetic profile of the agricultural product based on the analysis of the genetic data. Finally, the method comprises presenting, by the remote compute and store server, feedback based on the registration details and the genetic profile.

One embodiment of analyzing the genetic data of the present method comprises analyzing at least one genetic test sample that includes the one or more gene markers obtained from the agricultural product. An additional embodiment of analyzing the genetic test sample comprises identifying the agricultural product via a specific identifier, wherein the specific identifier comprises a bar code.

One embodiment of receiving the registration details defining the one or more characteristics of the agricultural product of the present method comprises receiving the registration details defining one or more characteristics of a crop or a livestock. One embodiment of presenting the feedback of the present method comprises presenting at least one of a nutritional recommendation, a breeding suggestion, a market valuation, a market forecast, and a lineage tracker.

One embodiment of receiving the genetic details of the present method comprises receiving at least one of genomic data, proteomic data, metabolomics data, and bioinformatics data. In addition, genomic data of the present disclosure may be at least one of DNA sequencing data, RNA sequencing data, or gene expression data.

The present method for managing agricultural products in an agricultural farm may also comprise analyzing, by the remote compute and store server, the genetic profile, and determining, by the remote compute and store server, feedback based on the analysis of the genetic profile. The method may further comprise performing, by the remote compute and store server, an automated function based on a result of the analysis of the genetic profile. The performing the automated function of the present method may comprise generating and transmitting at least one of (i) a notification to the user and (ii) a command to an actuator associated with a mechanized device of the agricultural farm. Generating the notification to the user of the present method may comprise generating at least one of an email, a text message, and an in-application notification. The method may further comprise presenting feedback to the user via a display of the computing device based on a result of the analysis of the genetic profile.

The present method for managing agricultural products in an agricultural farm may also comprise receiving, by the remote compute and store server, updated agricultural product data associated with the agricultural product. The method may further comprise analyzing, by the remote compute and store server, the genetic profile and the updated agricultural product data. In addition, the method may comprise determining, by the remote compute and store server, feedback based on the analysis of the genetic profile and the updated agricultural product data.

The present method may further comprise performing, by the remote compute and store server, an automated function based on a result of the analysis of the genetic profile and the updated agricultural product data. Performing the automated function of the present method may comprise at least one of (i) generating and transmitting a notification to the user and (ii) generating and transmitting a command to an actuator associated with a mechanized device of the agricultural farm. In addition, generating the notification to the user may comprise generating at least one of an email, a text message, and an in-application notification. The instant method may further comprise presenting feedback to the user via a display of the computing device based on a result of the analysis of the genetic profile and the updated agricultural product data.

A remote compute and store server for managing agricultural products in an agricultural farm of the present method may comprise a network communication circuit to (i) receive registration details from a user, wherein the registration details define one or more characteristics of an agricultural product to be analyzed and (ii) receive genetic data from a user, wherein the genetic data defines one or more gene markers of the agricultural product. The remote compute and store server of the present method may also comprise an agriculture analysis circuit to (i) analyze the genetic data and (ii) generate a genetic profile of the agricultural product based on the genetic data. In addition, the remote compute and store server of the present method may comprise a feedback determination circuit to present feedback based on the registration details and the genetic profile.

Analyzing the genetic data of the present remote compute and store server may comprise analyzing at least one genetic test sample that includes the one or more gene markers obtained from the agricultural product. The agricultural product of the present remote compute and store server to be analyzed may comprise a crop or a livestock.

Receipt of genetic data of the present remote compute and store server may comprise receiving at least one of genomic data, proteomic data, metabolomics data, and bioinformatics data. Genomic data of the remote compute and store server may comprise DNA sequencing data, RNA sequencing data, or gene expression data.

In one embodiment of the remote compute and store server, presenting the feedback may be to present at least one of a nutritional recommendation, a breeding suggestion, a market valuation, a market forecast, and a lineage tracker. An additional embodiment of the remote compute and store server may be to identify the agricultural product via a specific identifier, wherein the specific identifier comprises a bar code.

In another embodiment of the remote compute and store server, the agriculture analysis circuit may be to analyze the genetic profile, and determine feedback based on the analysis of the genetic profile. The feedback determination circuit may be to perform an automated function based on a result of the analysis of the genetic profile. Performing the automated function of the remote compute and store server may be to generate and transmit at least one of (i) a notification to the user and (ii) a command to an actuator associated with a mechanized device of the agricultural farm. Generation of the notification to the user of the remote compute and store server may comprise generating at least one of an email, a text message, and an in-application notification. The remote compute and store server of the present disclosure, wherein the feedback determination circuit may be to present feedback to the user via a display of the computing device based on a result of the analysis of the genetic profile.

The network communication circuit of the remote compute and store server of the present disclosure may be to receive updated agricultural product data associated with the agricultural product. The agriculture analysis module of the remote compute and store server may be to further analyze the genetic profile and the updated agricultural product data.

The feedback determination module of the remote compute and store server may be to determine feedback based on the analysis of the genetic profile and the updated agricultural product data. The feedback determination circuit may also be to perform an automated function based on a result of the analysis of the genetic profile and the updated agricultural product data, wherein to perform the automated function may be to generate and transmit at least one of (i) a notification to the user and (ii) a command to an actuator associated with a mechanized device of the agricultural farm. In addition, to generate the notification to the user may comprise generation of at least one of an email, a text message, and an in-application notification. Finally, the remote compute and store server of the present disclosure, wherein the feedback determination circuit may be to present feedback to the user via a display of the computing device based on a result of the analysis of the genetic profile and the updated agricultural product data.

### BRIEF DESCRIPTION OF THE DRAWINGS

The detailed description refers to the following figures in which:

FIG. 1 is a simplified block diagram of at least one embodiment of a system for analyzing genetic data of agriculture;

FIG. 2 is a simplified block diagram of at least one embodiment of a computing device of the system of FIG. 1;

FIG. 3 is a simplified block diagram of at least one embodiment of a remote compute and store server of the system of FIG. 1;

FIG. 4 is a simplified block diagram of at least one embodiment of an environment that may be established by the computing device of FIG. 2;

FIG. 5 is a simplified block diagram of at least one embodiment of an environment that may be established by the remote compute and store server of FIG. 3;

FIG. 6 is a simplified flow diagram of at least one embodiment of a method for analyzing genetic data of agriculture that may be executed by the remote compute and store server of FIGS. 3 and 5;

FIG. 7 is a simplified flow diagram of at least one embodiment of a method for analyzing updated agricultural product data of agriculture on that may be executed by the remote compute and store server of FIGS. 3 and 5; and

FIG. 8 is an illustrative graphical user interface (GUI) showing a dashboard view of the agriculture management and analysis software interface.

### DETAILED DESCRIPTION OF THE INVENTION

To meet the environmental and economic challenges described above, agriculture producers need an “easy to use,” effective tool that will help them analyze data associated with their agricultural farm and make decisions thereon. Such a tool may be usable to make decisions influencing an agricultural farm to achieve particular goal(s), such as the breeding of particular animals, tracking key feed changes affecting an animal's health, responding to climate changes, estimating the quality and valuation of animals, and/or nutritional planning. Quick and effective decision-making is generally a key to agricultural farm efficiency, but the requisite decision-making can be made possible with accurate interpretation and visualization of data. The present disclosure is directed to a technology for a user to analyze, interpret, and visualize genetic data of agriculture, as well as provide feedback (e.g., suggestions, improvements, projections, etc.) and/or perform an automated function.

A user of the present disclosure may include, but is not limited to, any entity involved in the lifecycle of agriculture (e.g., growing crops, rearing livestock, etc.). For example, an illustrative user of the present technology may include a manager of an agricultural farm (e.g., that produces crop and/or manages livestock), such as a farmer, a rancher, a breeder, a stocker, a buyer, a packer, a butcher, etc. Each user of the instant technology may add and/or update real-time genetic information regarding the agriculture at their particular point in the life cycle of the agriculture. Over time, the genetic information from each user in the life cycle of the agriculture is compiled into the instant technology to provide a cumulative, in-depth profile of genetic information for the agriculture.

An illustrative embodiment of the present disclosure allows each user in an agriculture life cycle to add genetic, environmental, health, and/or market information or data to the database of the present technology in order to provide a cumulative profile of genetic and/or market information for that particular type of agriculture (e.g., type of crop, type of animal, etc.). For example, a livestock animal, such as a cow, may pass through many different users over the course of its life (e.g., from breeding/birth to market). At the beginning of the cow's life, a user may be a breeder that adds genetic information to the present technology regarding the genetics of the cow's parents. Once the cow is sent from the breeder to a stocker, the stocker may then add genetic, environmental, health, and/or market information about the cow (e.g., types of feed and/or medicines) to the database of the present technology. The cow may then be sent to buyers at feedlots that will also add genetic, environmental, health, and/or market information about the cow to the database of the present technology. This process repeats throughout the life cycle of the cow, allowing each user in the food and/or supply chain of the cow to add specific genetic, environmental, health, and/or market information about the cow to the database of the present technology.

Over time, a cumulative profile of genetic, environmental, health, and/or market information and data about the cow is provided and available to users of the present technology. The profile provided in the present technology may be analyzed, reviewed, and interpreted at any point in the cow's life cycle. For example, data in the present technology is available to users moving forward and backwards through the supply chain, including the life cycle a respective livestock and/or crop. Thus, the present technology provides invaluable information that helps drive decisions, predict outcomes, and/or provide recommendations to one or more users involved in agriculture management.

It should be appreciated that a user may be one or more individuals, a company, or an organization that is a small- to mid-size producer, such as one who manages a herd of livestock ranging from about 1 to about 100 animals, from about 1 to about 1,000 animals, from about 100 to about 1,000 animals, from about 1 to about 50 animals, from about 10 to about 80 animals, from about 25 to about 75 animals, from about 1 to about 500 animals. It should be further appreciated that a user may be one or more individuals, a company, or an organization that is a large producer, such as one who manages a herd of livestock ranging from about 1 to about 10,000 animals, from about 500 to about 5,000 animals, from about 1,000 to about 100,000 animals, from about 5,000 to about 50,000 animals, from about 1,500 to about 8,000 animals, from about 2,500 to about 7,500 animals, from about 1,000 to about 5,000 animals, and more than about 1,500 animals.

As used herein, the term “agriculture” refers to crops, such as vegetables, fruits, flowers, and plants, or livestock, such as cows/bulls/cattle, pigs/hogs, poultry (e.g., chicken, turkey, etc.), goats, sheep, buffalo, horses, or any other type of livestock typically associated with agriculture. It should be appreciated that agriculture as used herein also comprises crops and/or livestock products, such as animal parts (internal or external parts) or plant parts, such as seeds, as and fruits, as well as eggs, dairy (milks and cheeses), poultry, and other agricultural products.

Information and/or data are added to the present technology by the user. A user may add, enter, and/or incorporate data and information into the database of the present technology either directly or indirectly. Direct data incorporation may occur by a user entering data or information about their agriculture directly into the database of the present technology, such as through manual and or automated data uploads or data dumps. Indirect data incorporation may occur by a third-party, such as a data analysis technician, entering data or information about the agriculture of a user into the database of the present technology, such as through manual and or automated data uploads or data dumps.

An illustrative embodiment of indirect data incorporation may begin with a genetic sample (e.g., hair, blood, semen, urine, or tissue) that is obtained or collected from a particular type of crop or livestock of interest. The genetic sample may be paired with a specific identifier, such as a bar code, a randomly generated alphanumeric code, or any type of specific identifier. The genetic sample may then be sent to a third-party, such as a genetic or genomic analysis center, where the genetic sample is analyzed for particular genes or traits of interest. The third-party genomic center may then manually or automatically upload the data for the genetic sample into the database of the present technology for availability and subsequent analysis by the user.

Genetic, environmental, health, and/or market information about crops or livestock that is added directly or indirectly to the database of the present disclosure ultimately provides a comprehensive and cumulative profile that is available for assessment and interpretation by users. For example, data and/or information incorporated into the present technology may be assessed by a user at an onsite location by scanning the bar code or specific identifier using a machine-readable storage medium, such as smartphone or a tablet.

It should be noted that an onsite location of the present disclosure may comprise any location where crops and/or livestock are grown, raised, farmed, fed, stored, or held for any period of time. For example, an exemplary embodiment of an onsite location includes, but is not limited to, a farm, a ranch, a breeder, a slaughterhouse, a feedlot, a store, a market, and a manufacturing and/or industrial facility.

One or more embodiments of the technology of the present disclosure may be implemented, in some cases, in hardware, firmware, software, or any combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device). Illustrative embodiments of a machine-readable storage medium include any type of computing device, such as, but not limited to, a computer, a laptop, a tablet, an e-book reader, a mobile/cell phone (including all operating systems), and a wearable computer device, such as an electronic watch, belt, or bracelet. The machine-readable storage medium is a means to analyze, interpret, and/or visualize genetic data implemented in the technology that ultimately may provide suggestions and recommendations regarding the agriculture managed by the user.

Technological advances in the field of genomics are changing the way producers, particularly small- to mid-size producers, manage their crops and livestock. New techniques such as genomics, proteomics, metabolomics, and bioinformatics are now being used by producers to generate data to better assess and make decisions regarding agriculture business. Genetic data implemented in the technology of the present disclosure may include any type of genotype or phenotype data, whether publicly or privately available. For example, genetic data may include any type of DNA, RNA, or protein (e.g., amino acid or peptide) mutation information, including but not limited to, data comprising Single Nucleotide Polymorphism (SNPs), Variable Number of Tandem Repeats (VNTRs), Copy Number Polymorphisms (CNPs or CNVs), Haplotypes or Linkage data, and customized, standard, or commercial genetic marker profiles.

Genomic tools and data of the present disclosure also include, but are not limited to testing results for genetic defects, paternity, and genomic-enhanced Expected Progeny Differences (EPDs). Further, genomic data can include results of platform-based tools, such as SNP arrays and gene-expression microarrays, as well as next-generation sequencing technologies. Genetic data of the present disclosure may promote marker-assisted agriculture management by comprising nutritional information (i.e., nutrigenomics), commercial valuation statistics, lineage/ancestry, and/or breeding information. For example, EPDs can help predict the genetic quality of future offspring, and thus, enables users to make informed, data-driven decisions to manage their animals, such as whether a particular animal should be kept for breeding or should be sold to the meat market. However, EPD data may be confusing or inconsistent with other genetic data.

TABLE 1
Genetic Data Interpretation and Inconsistency
DNA Test Results
MBV and EPD Marbling Panel Results

| | | Accuracy | | |
|---|---|---|---|---|
| | | or | | |
| Animal | MBV | Reliability | EPD | Accuracy |
| 1 | 0.10 | .20 | .30 | .17 |
| 2 | 0.40 | .22 | .20 | .15 |

For example, Table 1 shows the results of DNA testing of a marbling genetic marker panel on two Angus bulls (Animals 1 and 2). The test results are shown as a Molecular Breeding Value (MBV) or EPDs, and the associated accuracy (or reliability) of the MBV and EPD result, respectively. According to the MBV data, Animal 2 is the superior animal (0.4 MBV). To the contrary, according to the EPD results, Animal 1 is the superior animal (0.30 EPD). Though the EPD accuracies are lower, it is known in the art that the EPD value generally does a better job at predicting the total genetic merit of an animal as a parent. Moreover, it is also known in the art that MBV and EPD values are not calculated the same way, and thus, are not directly comparable. However, for users that are unfamiliar with interpreting and understanding genetic data, such as MBV and EPD values, the inconsistency between the values creates confusion and erroneous decision-making on behalf of the user when evaluating the results.

The technology of the present disclosure comprises a system that effectively combines genetic information into a robust agricultural operations and monitoring tool to help agriculture producers improve the performance and outcomes of their products. For example, in one embodiment, the technology of the present disclosure enables efficient genomic profiling of agriculture, such as an animal herd or flock. Thus, the technology also enables decision-making based on scientific or genomic data with a user-friendly approach such that the feedback (e.g., visual representation of data, suggestions, improvements, projections, etc.) can guide agriculture producers to make decisions regarding breeding, nutrition, health, and environment for their plants and/or animals over a period of time.

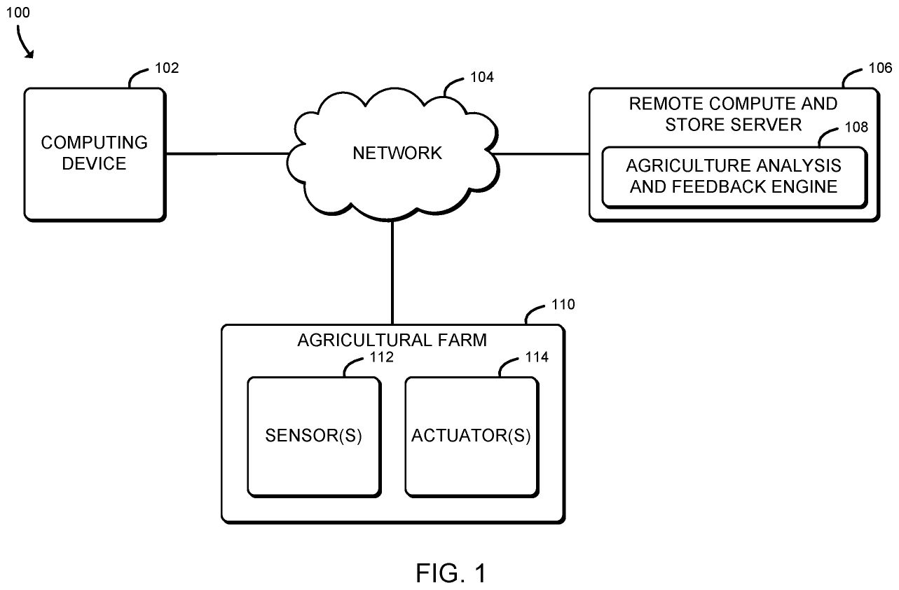

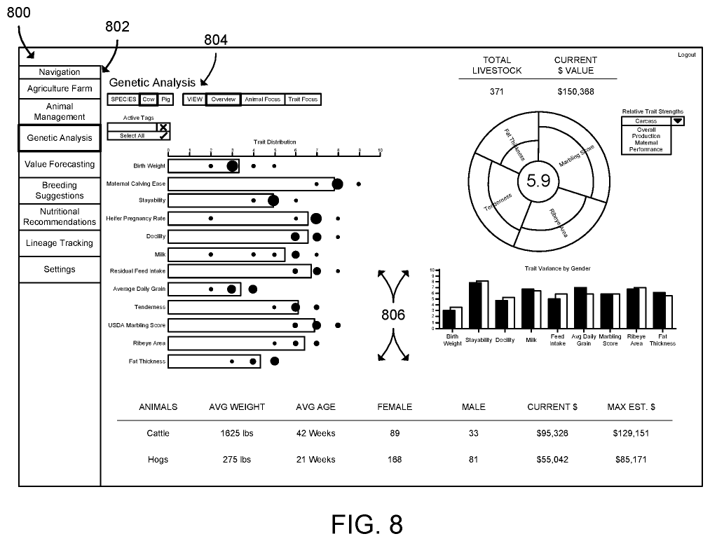

This platform technology (e.g., via a user interface of the application) can be usable to guide users through the interpretation of genomic data in order to enhance decision-making. Embodiments of this technology may include a mobile-friendly cloud-, web-, or subscription-based application that is accessible on a computing device (e.g., the computing device 102), as well as any thin or thick client application that may be installed or run from the computing device. The platform of the agriculture management and analysis system 100, as shown in FIG. 1, relies on various input data, such as basic customer profiles and/or an agriculture inventory system, as well as administrative portals, sample management tools, and/or a number of customer dashboard interfaces (see, e.g., FIGS. 8-11). Accordingly, such customer dashboard interfaces may allow for a user to input data, as well as user permissions, access roles, and logging on/off capabilities. For example, an account associated with the user may be accessible using one or more user credentials (e.g., a username, a password, a passphrase, biometric data, etc.) linked to the account. It should be appreciated that, in some embodiments, the instant technology may be available as an application or an “app” on a mobile computing device (e.g., a smartphone, a tablet, a wearable, etc.), or a software program on a stationary computing device (e.g., a desktop, a backend server, etc.).

The technology platform of the present disclosure comprises one or more, two or more, three or more, four or more, five or more, about five, about 5 to about 20, about 1 to about 25, or any number of categories of information that are necessary or convenient for the user. For example, the technology platform can deliver bioinformatics data that allows agriculture producers to do one or more or the following: 1) see the results of nutritional programs, 2) grade agriculture based on genetic merit, 3) identify crops and/or livestock that exhibit superior traits, and 4) guide decisions about crops and/or livestock (e.g., when to stop breeding livestock with low-quality traits, which animals to breed, how much to feed particular animals, identify what type of feed to distributed to particular livestock, which crops to plant and when, how much to fertilize/water, what type of fertilizer to apply to a particular crop, etc.).

The technology platform can also maintain key performance indicators (KPIs) corresponding to traits, in real time for producers. Such KPIs may include, but are not limited to, fertility, calving, production, etc. Additional KPIs of the present disclosure may include, but are not limited to reserve feed intake, average daily weight gain, tenderness, marbling score, percent choice, yield grade, fat thickness, ribeye area, heifer pregnancy rate, stayability, maternal calving ease, and docility.

Features of the present technology include, but are not limited to nutrition, valuation, forecasting, and parentage and/or lineage traceability. The technology platform enables producers to improve and/or change genetic profiles of their agriculture (e.g., crops being cultivated, animals being reared, etc.), as well as to select optimal breeding pairs, assess the value of agriculture, estimate the optimal time to keep agriculture before sales, and track lineage or crops or animals. While one embodiment of the present technology may not comprise any of the following five features (i.e., a custom kit), specifically, at least one, two, three, four, or all five of the following categories of information should be incorporated into the present technology:

Nutritional Assessment and Recommendations: Nutrigenomics is a field of study in its infancy, and it is the study of how diet influences gene expression and the health of animals, such as livestock. In particular, researchers work to understand how food components, such as nutrients and bioactive chemicals in foods and supplements, alter gene expression or the structure of the genome of an animal. Nutrigenomics is becoming more important, and will continue to advance as researchers develop a more thorough understanding of the relationship between nutrition, genetics, animal growth, and product quality.

The present technology comprises a key technical feature to visualize and interpret nutrigenomic (i.e., genomics and nutritional) data and information. For example, embodiments of the present system may enable: 1) processing large amounts of bioinformatics data that combines genetics and nutritional information, and 2) translation of the data into a format that is accurate, comprehensive, easy to use and understand by the user. The nutrigenomics program is able to distill a complex set of genetic and nutritional data derived from different statistical analyses, sources, or assumptions into results that are easily visualized and understood and readily available.

One embodiment of the nutrigenomics platform may comprise microarray or DNA chip technology results to allow screening of large numbers of genes, and to provide a user a detailed picture of the variation of the gene-expression patterns. It also offers a user insight into complex biological regulatory interactions, such as those between diet nutrients and genes. Many gene markers for phenotypic traits that producers typically use to improve their herds (e.g., fertility, calving, production, management and health) are known, and the nutrigenomic platform for genetic testing has already been validated by the industry. However, continuous advances in molecular genetics have led to the identification of multiple genes or markers associated with significant impact or effect on traits of interest in livestock.

Since livestock producers must utilize more effective and efficient decision-making at every level of their animal management, the present technology enables producers to evaluate their herd based on genetic merit and the effects of nutritional programs on the gene expression of their animals. Producers may use the nutrigenomics feature to improve feed efficiency (e.g., reduced food costs), to ensure that their animals are receiving the nutrients needed to improve growth rate under varying conditions, and to change feeding combinations and/or schedules based on learned information. For example, a producer could examine the potential impact of diet changes of animals to optimize health outcomes of sick or diseased animals.

The nutrigenomics features also provides information to aid or guide selection of certain food supplements by identifying the associated phenotypic traits that may be optimized or depressed given a specific food regimen. For the livestock food industry, the nutrigenomics feature may be useful for design, preparation, and marketing of livestock food supplements to producers. For producers, the nutrigenomics tool allows design of animal diet plans that promote optimal growth and development of the animal, and thus, enhanced marketability and profitability. Ultimately, the nutrigenomics feature of the present technology provides livestock producers a valuable tool to improve the nutritional quality, healthy weight, and economic value of an individual animal and/or an entire herd.

Genetic Profiles and Assessment: The genetic profiling and assessment feature of the present platform integrates genetic test data into a herd management tool for livestock producers of all sizes (e.g., small to large farms or animal herds). Visualization of a genetic profile data and information associated with a single animal, multiple animals or sub-groups of animals, or an entire herd can be made available via this feature. This feature may also include a profiling process for which producers will be able to grade their animals individually, as a sub-group, or the entire herd, and make decisions based on the genetic merit. A particular benefit of the genetic profiling feature is that it minimizes use or need for tables of data in the form of numbers, and provides intuitive labeling that is easy-to-use and to interpret (e.g., “Good,” “Average,” and “Bad”).

Breeding Traits and Suggestions: The breeding feature of the present technology provides a mechanism for the user to visualize and understand genetic profiles of their animals in order to identify and select good breeders or specific cross-breeding strategies. The breeding feature may also provide predictions and expectations regarding progeny traits, including health, wean weight, residual feed intake, average daily (weight) gain, tenderness, marbling (score), yield grade, fat thickness, ribeye (area), heifer pregnancy rate, stayability, myostatin, quality grade, and docility. This feature may also provide predictive data regarding maternal function traits of animals (e.g., pregnancy rate, milk production, and maternal calving ease.

Finally, the breeding technology feature uses genetic data and information to provide matching services to owners. Embodiments of this matching services feature may include options to match up bulls and cows or hogs and sows, respectively, in preparation for artificial insemination (AI). Thus, the breeding feature enables producers to improve genetic profiles of their animals by selecting optimal breeding pairs to produce progeny with valuable and/or improved phenotypes or traits.

Valuation and Forecasting: The valuation feature of the present technology comprises a collection and organization of relevant genomic data incorporated with available market data about the agriculture products, and enables easy visualization of a genetic profile, performance qualities, and marketability. One embodiment of the valuation feature provides a user real-time access to market prices (e.g., feed prices, sale barn data), and predictive changes in market prices, such that the user may determine an optimal sale price or target date of sale for animals. The valuation tool analyzes market and genetic profile data to make predictive estimates on value trends, such as price increase or decrease, weight increase or decrease, or stagnation. Based on the market and genetic data, the valuation feature provides estimates on optimal sale date in order to maximize profitability. The valuation feature is easy to understand, easy to interpret, and easy for the producers to assess and make decisions regarding the value of livestock and to estimate the optimal time to keep livestock before sale.

Lineage Tracking: The lineage tracking feature of the present technology comprises DNA testing results to maintain an inventory of family lines and heritage. The lineage tracking features enables a user to visualize and understand a past market and to predict animal genetics and value more accurately in the future.

In addition, one or more custom traits may be added to one or more of the platform categories of the present technology described above. For example, any trait that is economically important or that impacts agriculture value and sustainability, or that is requested by a user, may be added to the present technology. A custom kit is the present technology comprising none of the five feature categories described above.

The technology platform utilizes genetic technology (e.g., DNA or RNA sequencing) to measure genetic data regarding individual animal feed and water intake on a large group of animals, such as cows or steers. Such genetic data may include tissue collection, RNA isolation, genomic library construction, sequencing read quality assessment, RNA sequencing read processing, statistical analysis of RNA sequencing gene expression, identifying gene transcripts as predictive classifiers of growth and carcass traits, etc. Along with feed and water intake, the genetic data can be analyzed to result in data performance metrics. The data performance metrics may then be further analyzed to assess the predictive capability of genetic metrics to support data-driven decisions included in a livestock management program.

In an illustrative data gathering sequence of the genetic data collection, a total of 120 crossbred beef steer calves, at least 240 days of age, with an approximate initial body weight of 280 kg, were utilized in a 91-day feed and water intake trial. Within groups, all animals were blocked by weight and randomly allocated to one of four pens (12.2×30.5 m) with 30 animals per pen. Each pen provides 186.5 m² of shade and is equipped with an Insentec feed and water intake system comprised of six feed bunks and one water trough. For the cattle on trial, the study began with a 21-day acclimation period following arrival.

After the acclimation period, a 70-day feed and water intake trial was conducted to assess relationships between feed and water intake and genetics, and the health, performance, and behavior of low feed vs. high feed and water intake animals. To be in compliance with feed intake guidelines outlined by the Beef Improvement Federation (BIF, 2012), animal weights were measured at least every 14 days. At the conclusion of the growing and a subsequent 70-day water restriction phase, animals continued to the finishing phase under normal management, and carcass quality attributes (e.g., hot carcass weight, kidney/pelvic/heart fat percentage, 12th rib backfat, ribeye area, marbling score, USDA quality grade, and USDA yield grade) were collected at harvest.

In another illustrative data gathering sequence, 20 steers were randomly selected from the steers on feed for RNA collection at the end of the acclimation period (on or after day 21). Three milliliter (3 ml) samples of whole blood were collected into Tempus™ tubes (Ambion, Austin, TX) before the end of the feed and water intake trial and cooled on ice. Ear notches (tissue samples) were also collected and flash-frozen. All blood and tissue samples were shipped overnight on ice packs for RNA extraction.

After cattle were harvested, feeding data, animal weights, and carcass data for the selected animals (n=20) was also provided for analysis. Total RNA was isolated from blood and tissue samples using the Tempus™ Spin RNA Isolation Kit (Ambion, Austin, TX, USA) according to the manufacturer's protocol. An ND-1000 spectrophotometer (Nano-Drop Technologies, Wilmington, DE, USA) was used to quantify RNA concentrations. The globin transcripts (HBA and HBB) were reduced using an RNase H-based globin reduction method (Affymetrix GeneChip GR Protocol) for reduction of globin mRNA. To determine the quality of the RNA both prior to and following globin reduction, RNA samples were assayed for their 28S to 18S rRNA ratio using an Agilent 2100 Bioanalyzer software (Agilent Technologies, Inc., Santa Clara, CA, USA). RNA integrity numbers (RIN) were determined using a Bioanalyzer to assess both pre- and post-globin reduction.

Total RNA was also isolated from the 20 cow blood samples using the Tempus™ Spin RNA Isolation Kit (Ambion, Austin, TX, USA) according to the manufacturer's protocol. Total RNA was isolated with Trizol™ according to the manufacturer's protocol, and RNA integrity and quantitation was carried out as described in paragraph for globin depletion.

Genomic library construction of the cow samples was conducted with the TruSeq™ library kit (Illumina, Inc., San Diego, USA) according to the manufacturer's protocol. Sequencing was performed with an Illumina HiSeq machine using 100 cycles and the paired-end read methodology, as described by the manufacturer (Illumina, Inc., San Diego, USA). Ten samples were allocated to one lane, such that fixed effects were confounded with lane effects, such that the statistical power to detect informative transcripts was maximized. Initial processing of reads from the HiSeq machine was performed with the Illumina CASAVA (v1.8) software.

All sequence reads were trimmed using the Sickle software to remove poor quality sequence and ensure no adaptor remnants remain with any read. Sequence reads were confirmed with FASTQC software (available at: http://www.bioinformatics.babraham.ac.uk/projects/fastqc/) to determine if the sequences had sufficient read quality (e.g., reasonable read length, GC content, low percentage of repeated sequence) for downstream analyses.

To produce a common set of transcript coordinates for all test samples, sequence reads for each sample was mapped to the UMD3.1 reference assembly using tophat/bowtie2. Sequence alignment files from all samples were merged into a single BAM alignment file and cufflinks were used to define a set of common transcript coordinates for all samples. For each sample, any reads that aligned to the HBB and HBA reference sequences were removed before HiSeq analysis using the common transcript coordinates to determine discrete counts for each transcript.

Association between individual transcripts with growth and carcass traits, were first identified using test individual taxa with a linear model, which included fixed effects. The trimmed mean of M values (TMM) normalization procedure from the edgR package in R was utilized to normalize RNA transcript counts based on the full set of genome-wide counts. This procedure also involved adjustment for the variation in transcript library size seen across samples. Normalized counts were log 2 transformed to obtain the resulting scaled values used for analysis. Covariates for pre- and post-globin-reduction RIN and 5′-3′ transcript read skewness were considered to be included based on model selection using Aikake information criterion (AIC) comparisons to select the best model, and thus, correct for mRNA quality.

Statistical significance was determined using false discovery rate (FDR) correction. Given that transcripts behaved as groups (i.e., are correlated), transcript structures were also identified by correlation-based analyses across the animals, looking specifically for groups of transcripts that co-varied or that were antagonistic within growth and carcass traits. Principal Component Analysis (PCA) and Linear Discriminant Analysis (LDA) were performed on the covariance matrices to measure group association (high versus low growth). If significant differentiation of the transcripts between growth and carcass traits was observed, the actual structures and sets of synergistic and antagonistic transcripts was defined by network analysis using stringent correlation thresholds. Other statistical methods may be used for analysis of transcriptome data to identify clusters of genes that discriminate traits such as, for example, the Random Forest Classifier methodology.

Transcripts that are associated with growth and carcass traits and identified via the methodology described herein, demonstrate feasibility for the present technology's potential to predict, to some extent, variation in traits prior to the collection of that trait information. Further, identification of a set of gene transcripts capable of explaining significant variation in genetic traits of economic importance in animal is highly valuable. In particular, a set of gene transcripts has more discriminating power than a single transcript, and as such, would be able to predict more phenotypic variation expected in animals.

Therefore, identification of a panel or subset of gene transcripts ranging from about 10 to about 50, from about 5 to about 100, from about 25 to about 75, and from about 1 to about 40, may be used to predict the performance of cattle in the feedlot. It is also possible use variation in gene expression data to optimize animal nutrition in the future. For example, using the nutrigenomics feature of the present technology, a user is able to classify animals (e.g., cattle) with respect to their ability to positively respond to nutrient X (e.g., higher protein content, increased energy, etc.) or negatively respond to nutrient X (e.g., slower growth rate or require more days on feed, etc.).

As described previously, the technology platform includes several primary categories of genetic data information including valuation, lineage tracing, nutrigenomics, breeding, and genetic profiling features. Each featured category may incorporate a collection of relevant genomic data, algorithm development and testing, and implementation of visualization capabilities of a genetic profile associated with an animal or animal herd. As described previously, a user (e.g., a manager of an agricultural farm that produces crop and/or manages livestock) may access the technology platform via a customer account, which may be encrypted with prompts that require proper submission of credential, such as a username, a password, a passphrase, biometric data, etc.

Accordingly, the user may input registration details of one or more agricultural products, such as any details of various crops and/or livestock animals that the user may want to retrieve data for. The registration details may include any type of data relative to the crops and/or livestock, including, but not limited to type of animal, breed or gender of animal, ear tag number, location, date of birth, pen number, siblings, traits, etc. It should be appreciated that, in some embodiments, the input of registration details may be performed via a batch upload technology to reduce time and increase the accuracy of data information and analysis. Accordingly, in such embodiments, the registration details may be formatted in a particular format required by the technology platform and the technology platform may be configured to parse the registration details based on the format. The registration details may then be analyzed for genetic, nutritional, and market analysis.

Additionally, custom sample collection kits are may be provided to the user in order to obtain genetic test samples from the animals. Genetic test samples may be in any form from which DNA, RNA, and/or proteins may be extracted, including but not limited to hair, blood, semen, urine, or tissue. A semen sample from male animals may be preferred if breeding analysis or further analyses regarding breeding are desired by the user. Used collection kits may then be analyzed in order for genetic profiles of the animals to be prepared.

Genetic profile results may include any and all raw, statistically analyzed, or statistically significant results generated by the data sample submitted by user. Genetic profile results also comprise any results generated by the data sample submitted by user that is corrected for technical, physical, or statistical errors. In some embodiments, genetic profile results may be entered by a user and/or by a third party vendor (e.g., for controlled access) to a remote compute and store server 106 of an agriculture management and analysis system 100. For example, a user or a third party vendor may input the genetic profile results into a computing device 102 communicatively coupled to the remote compute and store server 106 via a network 104.

In use, as described in further detail below, the genetic profile results, as well as the registration details of the agricultural products, can be processed by an agriculture analysis and feedback engine 108 of the remote compute and store server 106 to determine feedback to present to a user, such as via an interface (e.g., a display) of the computing device 102. Such feedback may be in the form of a visual representation of data, such as charts, figures, graphs, numbers, codes, and/or any type of visual representation of data that is helpful to the user. The feedback may additionally provide recommendations, suggestions, projections, etc. to the user as to how to manage their agriculture. For example, the feedback may be provided in the form of breeding recommendations, valuation numbers and/or estimates, fertilizer/water recommendations, feed/dietary suggestions, etc.

In use, subsequent to the genetic profile results having been input, a user may access their account to review the analysis and interpretation of the genetic profile data as determined by the agriculture analysis and feedback engine 108. It should be appreciated that the user may need to periodically add, update, or otherwise revise information related to the agriculture that is being managed and analyzed by the agriculture analysis and feedback engine 108. For example, in an embodiment in which the agriculture includes livestock, the user may need to change livestock information related to the animal itself, breeding and/or nutrition information, KPI (e.g., weight, sickness, etc.), or herd detailed information. Further, the user may access and refer to information provided by the agriculture analysis and feedback engine 108 in preparation of making certain decisions (e.g., breeding, feeding, marketing, and/or sales decisions).

In in illustrative embodiment, the agriculture analysis and feedback engine 108 described herein can be used to improve livestock producers' ability to make decisions based on the integration and understanding of genetic and nutritional data. To do so, the agriculture analysis and feedback engine 108 may allow users to access real-time information about their agriculture. In such embodiments, the real-time information may be collected from one or more sensors (e.g., the sensors 112 of the agricultural farm 110). Further, users may use the agriculture management and analysis system 100 as a tool for personal genomic management information systems. Accordingly, the agriculture management and analysis system 100 may allow the user to track key metrics of the agriculture management and analysis system 100 by visualizing the data associated with their agriculture, keeping track of key performance indicator of genetic traits, planning and monitoring livestock nutrition, deciding upon breeding logistics, keeping accurate inventories on their livestock for full DNA traceability, and making sound breeding selections/predictions based on the analysis performed by the agriculture analysis and feedback engine 108.

Such tracking of key metrics can be interpreted to either manually or automatically take certain actions on the agriculture being analyzed to increase productivity and value, improve environmental conditions, reduce loss, and produce healthier, more profitable agriculture (e.g., crops and/or livestock). Accordingly the users can produce healthier livestock and make precise decisions when it comes to breeding and feeding their herds, maximizing the use of water and land resources, and providing healthy agricultural products to consumers. The agriculture analysis and feedback engine 108 can also provide users with the ability to put the right value on the agricultural products, as well as help improve the market price of the agricultural products, which can lead to a more profitable agricultural farm 110.

As the population grows, the environmental impact of agriculture is expected to increase proportionally. In an illustrative example, the agriculture analysis and feedback engine 108 can be used to help users of the agriculture management and analysis system 100 to design a selective breeding plan that will be successful in terms of yield and production efficiency, which may in turn allow for a better use of land and water resources, including areas that are not suitable for agricultural purposes due to environmentally unfavorable conditions. Accordingly, livestock breeding may be influenced by various attributes such as product quality, animal welfare, disease resistance, environmental impact reduction, and implementation of molecular genetic tools that can impact the agriculture of the agriculture management and analysis system 100.

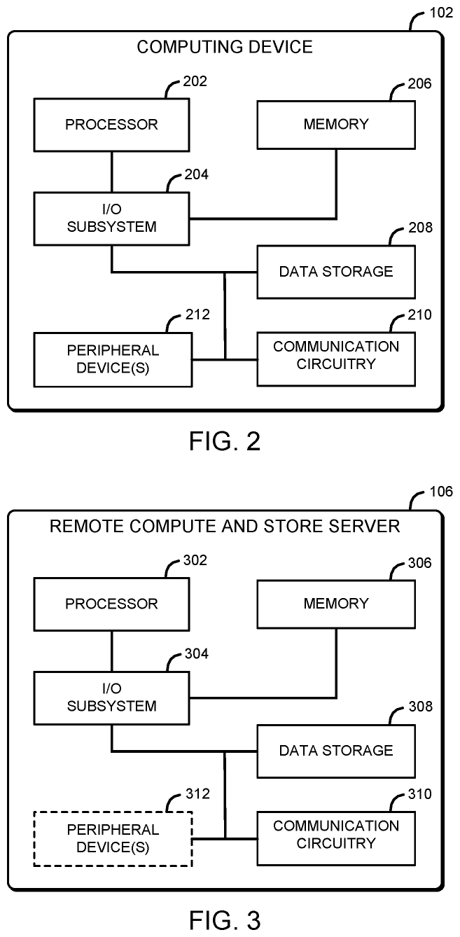

The computing device 102 may be embodied as any type of computation or computing device capable of performing the functions described herein, including, without limitation, a computer, a desktop computer, a smartphone, a workstation, a laptop computer, a notebook computer, a tablet computer, a mobile computing device, a wearable computing device, a network appliance, a web appliance, a distributed computing system, a processor-based system, and/or a consumer electronic device. As shown in FIG. 2, the illustrative computing device 102 includes a processor 202, an input/output (I/O) subsystem 204, a memory 206, a data storage device 208, communication circuitry 210, and one or more peripheral devices 212. Of course, in other embodiments, the computing device 102 may include other or additional components, such as those commonly found in a computing device (e.g., input/output devices, etc.). Additionally, in some embodiments, one or more of the illustrative components may be incorporated in, or otherwise form a portion of, another component. For example, in some embodiments, the memory 206, or portions thereof, may be incorporated in the processor 202.

The processor 202 may be embodied as any type of processor capable of performing the functions described herein. The processor 202 may be embodied as a single or multi-core processor(s), digital signal processor, microcontroller, or other processor or processing/controlling circuit. The I/O subsystem 204 may be embodied as circuitry and/or components to facilitate input/output operations with the processor 202, the memory 206, and other components of the computing device 102. For example, the I/O subsystem 204 may be embodied as, or otherwise include, memory controller hubs, input/output control hubs, integrated sensor hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 204 may form a portion of a system-on-a-chip (SoC) and be incorporated, along with the processor 202, the memory 206, and other components of the computing device 102, on a single integrated circuit chip.

The memory 206 may be embodied as any type of volatile or non-volatile memory or data storage capable of performing the functions described herein. In operation, the memory 206 may store various data and software used during operation of the computing device 102 such as operating systems, applications, programs, libraries, and drivers. The memory 206 is communicatively coupled to the processor 202 via the I/O subsystem 204, which may be embodied as circuitry and/or components to facilitate input/output operations with the processor 202, the memory 206, and other components of the computing device 102. For example, the I/O subsystem 204 may be embodied as, or otherwise include, memory controller hubs, input/output control hubs, integrated sensor hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 204 may form a portion of a system-on-a-chip (SoC) and be incorporated, along with the processors 202, the memory 206, and other components of the computing device 102, on a single integrated circuit chip.

The data storage device 208 may be embodied as any type of device or devices configured for short-term or long-term storage of data such as, for example, memory devices and circuits, memory cards, hard disk drives, solid-state drives, or other data storage devices. The data storage device 208 may include a system partition that stores data and firmware code for the computing device 102. The data storage device 208 may also include an operating system partition that stores data files and executables for an operating system of the computing device 102.

The communication circuitry 210 may be embodied as any communication circuit, device, or collection thereof, capable of enabling communications over the network 104 between the computing device 102 and the remote compute and store server 106. The communication circuitry 210 may be configured to use any one or more communication technologies (e.g., wired and/or wireless communication technologies) and associated protocols (e.g., Ethernet, Bluetooth®, Wi-Fi®, WiMAX, etc.) to effect such communication.

The peripheral devices 212 may include any number of peripheral or interface devices, such as a display, a touchscreen, speakers, a microphone, a printer, additional storage devices, and so forth. The particular devices included in the peripheral devices 212 may depend on, for example, the type and/or intended use of the computing device 102. Additionally or alternatively, the peripheral devices 212 may include one or more ports, such as a USB port, for example, for connecting external peripheral devices to the computing device 102.

The remote compute and store server 106 may be embodied as any type of computation or computer device capable of performing the functions described herein, including, without limitation, a server (e.g., stand-alone, rack-mounted, blade, etc.), a network appliance (e.g., physical or virtual), a web appliance, a distributed computing system, a processor-based system, a multiprocessor system, a smartphone, a mobile computing device, a tablet computer, a laptop computer, a notebook computer, and/or a computer.

Similar to the computing device 102, the illustrative remote compute and store server 106 includes a processor 302, an input/output (I/O) subsystem 304, a memory 306, a data storage device 308, communication circuitry 310, and, in some embodiments, one or more peripheral devices 312 (see FIG. 3). As such, further descriptions of the like components are not repeated herein for clarity of the description with the understanding that the description of the corresponding components provided above in regard to the computing device 102 of FIG. 2 applies equally to the corresponding components of the remote compute and store server 106 of FIG. 3. Of course, in other embodiments, the remote compute and store server 106 may include other or additional components, such as those commonly found in a computing device.

The illustrative remote compute and store server 106 includes an agriculture analysis and feedback engine 108. The agriculture analysis and feedback engine 108 may be embodied as any software, firmware, hardware, or combination thereof capable of performing the functions described herein. In particular, the agriculture analysis and feedback engine 108 is configured to support accessing data (e.g., received registration details, genetic data, analysis results, etc.) and executing code to analyze the accessed data, as well as the other functions described herein.

The network 104 may be embodied as any type of wired or wireless communication network, including cellular networks (e.g., Global System for Mobile Communications (GSM), 3G, Long Term Evolution (LTE), Worldwide Interoperability for Microwave Access (WiMAX), etc.), digital subscriber line (DSL) networks, cable networks (e.g., coaxial networks, fiber networks, etc.), telephony networks, local area networks (LANs) or wide area networks (WANs), global networks (e.g., the Internet), or any combination thereof. Additionally, the network 104 may include any number of network devices (not shown), such as routers, access points, switches, etc. as needed to facilitate communication between the computing device 102 and the remote compute and store server 106.

The agricultural farm 110 may be any area of land or structure usable to cultivate animals, plants, and/or any other type of farmable agriculture in which the primary objective is to produce food (e.g., livestock, crops, etc.). The illustrative agricultural farm 110 includes one or more sensors 112 and one or more actuators 114.

The one or more sensors 112 may include any type of sensor device capable of gathering data and providing the gathered data to the remote compute and store server 106 for analysis. In some embodiments, the one or more sensors 112 may include a measurement sensor (e.g., temperature, mass, volume, acoustics, light, flow, pressure, speed, particular matter, etc.), a location sensor (e.g., global positioning device (GPS) tag, a near-field communication (NFC) tag, etc.), an image sensor (e.g., an infrared (IR) sensor, a camera sensor, etc.), a motion sensor (e.g., passive IR, microwave, ultrasonic, radio wave, etc.), an actuator position sensor, and/or any other type of sensor capable of gathering data usable by the agriculture analysis and feedback engine 108 to provide feedback (e.g., suggestions, improvements, projections, etc.) to a user and/or perform an automated function in response thereto. In some embodiments, the sensors 112 may be interconnected via a mesh network (e.g., a massively interconnected network) in which a number of the sensors (e.g., implemented as internet of things (IoT) devices) are in communication with each other (i.e., interconnected) via network links (e.g., radio links), all of which are not shown in FIG. 1 to simplify the figure and preserve clarity.

The one or more actuators 114 may include any type of actuator device (e.g., a valve, a switch, etc.) capable of performing a function in response to having received a command. Such functions may include opening/closing a cover, starting/stopping a mechanized device, etc. In some embodiments, the one or more actuators 114 may be remotely controlled by the agriculture analysis and feedback engine 108 and/or via a user by way of the computing device 102 to perform a particular action or other function as described herein, such as may be performed in response to the analysis performed by the agriculture analysis and feedback engine 108 of the data collected from the one or more sensors 112.

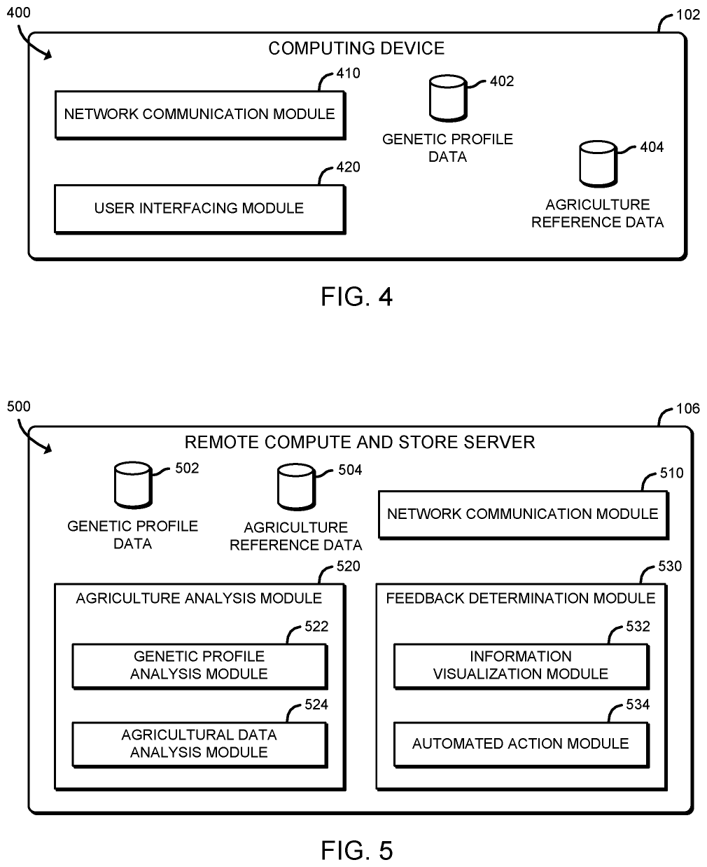

Referring now to FIG. 4, in use, the computing device 102 establishes an environment 400 during operation. The illustrative environment 400 includes a network communication module 410 and a user interfacing module 420. Each of the modules, logic, and other components of the environment 400 may be embodied as hardware, software, firmware, or a combination thereof. For example, each of the modules, logic, and other components of the environment 400 may form a portion of, or otherwise be established by, the processor 202 and/or other hardware components of the computing device 102. As such, in some embodiments, one or more of the modules of the environment 400 may be embodied as circuitry or a collection of electrical devices (e.g., network communication circuitry 410, user interfacing circuitry 420, etc.).

In the illustrative environment 400, the computing device 102 includes genetic profile data 402 and agriculture reference data 404, each of which may be accessed by the various modules and/or sub-modules of the computing device 102. It should be appreciated that the computing device 102 may include other components, sub-components, modules, sub-modules, and/or devices commonly found in a computing device, which are not illustrated in FIG. 4 for clarity of the description.

The network communication module 410, which may be embodied as hardware, firmware, software, virtualized hardware, emulated architecture, and/or a combination thereof as discussed above, is configured to facilitate inbound and outbound network communications (e.g., network traffic, network packets, network flows, etc.) to and from the computing device 102. To do so, the network communication module 410 is configured to receive and process network packets from other computing devices (e.g., the remote compute and store server 106) and prepare and transmit network packets to other computing devices (e.g., the remote compute and store server 106). For example, the network communication module 410 is configured to transmit network packets containing input from the user to the remote compute and store server 106 and receive network packets containing feedback for display to the user from the remote compute and store server 106.

The user interfacing module 420, which may be embodied as hardware, firmware, software, virtualized hardware, emulated architecture, and/or a combination thereof as discussed above, is configured to facilitate the input of data between a user and the computing device. In some embodiments, the user interfacing module 420 may be configured to interface with a display (not shown) of the computing device 102, such as by displaying one or more graphical user interfaces (GUIs) for receiving input and displaying feedback. As described previously, the input data may include data related to genetic profiles, which may be saved in the genetic profile data 402, as well as data related to the agriculture outside the scope of the genetic profiles, which may be saved in the agriculture reference data. In some embodiments, the user interfacing module 420 may be executed as a web-based thin client and/or a locally installed thick client.

Referring now to FIG. 5, in use, the remote compute and store server 106 establishes an environment 500 during operation. The illustrative environment 500 includes a network communication module 510, an agriculture analysis module 520, and a feedback determination module 530. Each of the modules, logic, and other components of the environment 500 may be embodied as hardware, software, firmware, or a combination thereof. For example, each of the modules, logic, and other components of the environment 500 may form a portion of, or otherwise be established by, the processor 302, the agriculture analysis and feedback engine 108, and/or other hardware components of the remote compute and store server 106. As such, in some embodiments, one or more of the modules of the environment 500 may be embodied as circuitry or a collection of electrical devices (e.g., network communication circuitry 510, agriculture analysis circuitry 520, and feedback determination circuitry 530, etc.).

In the illustrative environment 500, the remote compute and store server 106 includes genetic profile data 502 and agriculture reference data 504, each of which may be accessed by the various modules and/or sub-modules of the remote compute and store server 106. It should be appreciated that the remote compute and store server 106 may include other components, sub-components, modules, sub-modules, and/or devices commonly found in a computing device, which are not illustrated in FIG. 5 for clarity of the description.

The network communication module 510, which may be embodied as hardware, firmware, software, virtualized hardware, emulated architecture, and/or a combination thereof as discussed above, is configured to facilitate inbound and outbound network communications (e.g., network traffic, network packets, network flows, etc.) to and from the remote compute and store server 106. To do so, the network communication module 510 is configured to receive and process network packets from other computing devices (e.g., the computing device 102) and prepare and transmit network packets to other computing devices (e.g., the computing device 102). For example, the network communication module 510 is configured to receive network packets containing input from the user from the computing device 102 and transmit network packets containing feedback for display to the user to the computing device 102.

The agriculture analysis module 520, which may be embodied as hardware, firmware, software, virtualized hardware, emulated architecture, and/or a combination thereof as discussed above, is configured to analyze the received genetic profiles and registration details. Accordingly, to do so, the illustrative agriculture analysis module 520 includes a genetic profile analysis module 522 for analyzing the genetic profiles and an agriculture data analysis module 524 for analyzing agricultural data (e.g., registration details, data collected from the one or more sensors 112, genetic data, results of previously performed analyses, etc.).

In some embodiments, the genetic profiles may be stored in the genetic profile data 502. In such embodiments, the genetic profile analysis module 522 may be configured to retrieve one or more genetic profiles from the genetic profile data 502 on which to perform an analysis. Additionally, in some embodiments, the agricultural data may be stored in the agricultural reference data 504. In such embodiments, the agricultural data analysis module 524 may be configured to retrieve one or more agricultural references from the agricultural reference data 504 to perform an analysis thereon.

The feedback determination module 530, which may be embodied as hardware, firmware, software, virtualized hardware, emulated architecture, and/or a combination thereof as discussed above, is configured to determine feedback based on analysis of aggregated data (e.g., stored in the genetic profile data 502 and/or the agricultural reference data 504), such as may be performed by the agriculture analysis module 520. To do so, the illustrative feedback determination module 530 includes an information visualization module 532 and an automated action module 534. The information visualization module 532 is configured to compare and further analyze at least a portion of the aggregated data to present the analyzed data in a format usable to visualize the analyzed data.

For example, the visual representation of the analyzed data may be presented in any format usable to identify certain attributes of the data, such as a chart (e.g., a pie chart, a line chart, a bar graph, etc.). Accordingly, the information visualization module 532 may be configured to format the feedback such that the feedback can be transmitted to the computing device 102 (e.g., via the network communication module 510) for display on the computing device 102 (e.g., via an output device, such as a display, of the computing device 102). As such, a user may track visual representations of the analyzed data (e.g., environmental, health, genetic, performance data, etc.) of agriculture over their life cycle. In an illustrative example, the user can track visual representations of the analyzed data of cattle when moving through different chains from calf breeders, to stockers, to feedlots, to packers, to stores, etc. To do so, a globally unique barcode may be assigned to the agriculture (e.g., a particular animal of livestock, a particular section of crop, etc.), for which the output data can then be fed back into the agriculture management and analysis system 100 such that the selected agriculture's life cycle can be aggregated with other data to improve the analysis. For example, such data can be fed back into a genetic performance model to refine future estimations based on similar genetic attributes of the agriculture type.

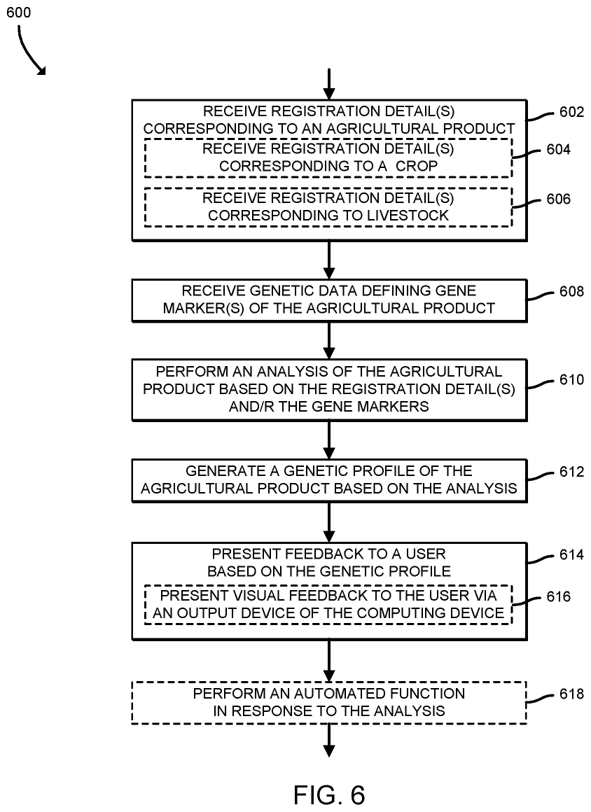

Referring now to FIG. 6, in use, the remote compute and store server 106 (e.g., via the agriculture analysis and feedback engine 108) may execute a method 600 for analyzing genetic data of one or more agricultural products. As described previously, the agricultural products may include various types of crops and/or livestock. The method 600 begins with block 602, in which the remote compute and store server 106 receives one or more registration details corresponding to an agricultural product. For example, in some embodiments, in block 604, the remote compute and store server 106 may receive registration details corresponding to a crop (e.g., a variant of corn, soybean, etc.). In another example, in some embodiments, in block 606, the remote compute and store server 106 may receive registration details corresponding to livestock (e.g., a horse, a pig, a cow, etc.).

In block 608, the remote compute and store server 106 receives genetic data defining one or more gene markers of the agricultural product. As described previously, many gene markers that producers typically use to improve their agricultural product (e.g., fertility, calving, production, management, and health) are known, and the nutrigenomic platform for genetic testing has already been validated by the industry. In block 610, the remote compute and store server 106 performs an analysis of the agricultural product based on the registration details received in block 602 and/or the gene markers received in block 608.

In block 612, the remote compute and store server 106 generates a genetic profile of the agricultural product based on the analysis performed at block 610. As described previously, the genetic profile may include any and all raw, statistically analyzed, or statistically significant results generated by the data sample submitted for a particular agricultural product. As also described previously, the genetic profile results may also comprise any results generated by the data sample submitted for a particular agricultural product that is corrected for technical, physical, or statistical errors.

In block 614, the remote compute and store server 106 presents feedback to the user based on the genetic profile generated in block 612. For example, in block 616, in some embodiments, the remote compute and store server 106 may present the feedback by transmitting data to a computing device (e.g., the computing device 102) for display on the computing device. It should be appreciated that, in some embodiments, such as in those embodiments in which the feedback is being displayed by a thick client, the data usable to generate the visual representation may be transmitted to the computing device on which the visual representation is to be prepared, rendered, and displayed. It should be further appreciated that, in other embodiments, such as those embodiments in which the feedback is being displayed by a thin client accessed by the computing device (e.g., via a web server on the remote compute and store server 106), the visual representation may be prepared by the remote compute and store server 106 and the data related thereto may be transmitted to the computing device for rendering and display.

Additionally, in some embodiments, in block 618, the remote compute and store server 106 may initiate an automated function in response to the analysis. It should be appreciated that, in some embodiments, the automated function may be performed in response to a trigger, a setting, or an instruction implemented by a user of a computing device on which the feedback is displayed (e.g., the computing device on which the agriculture management and analysis system 100 is managed). For example, in an illustrative embodiment, the remote compute and store server 106 may transmit a command to one or more actuators 114 of the agricultural farm 110 to perform a particular action. In another illustrative embodiment, the remote compute and store server 106 may transmit a notification (e.g., a text message, an email, a fax, an in-app notification, etc.) to the user that includes the feedback information and/or a hyperlink directed thereto.

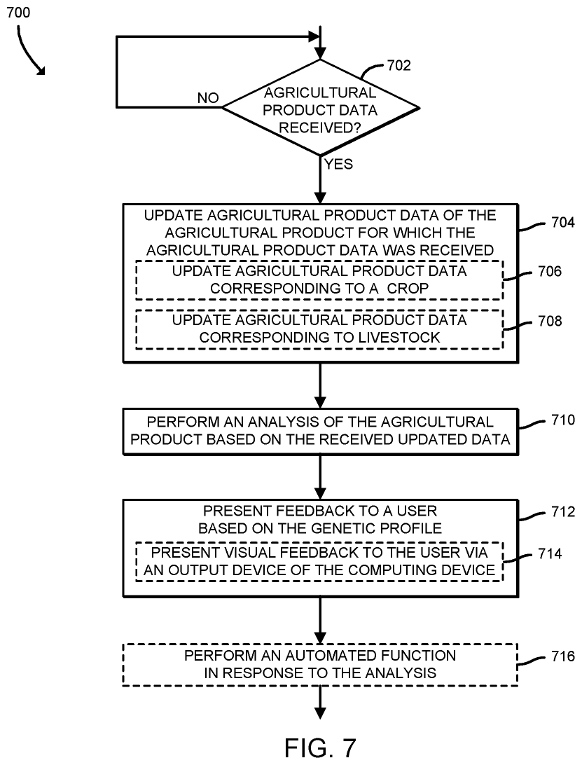

Referring now to FIG. 7, in use, the remote compute and store server 106 (e.g., via the agriculture analysis and feedback engine 108) may execute a method 700 for analyzing genetic data of one or more agricultural products. As described previously, the agricultural products may include various types of crops and/or livestock. The method 700 begins with block 702, in which the remote compute and store server 106 determines whether information (e.g., an update) for an agricultural product has been received. It should be appreciated that the updated information may be input by a user (e.g., via an interface of the computing device 102 and data transmitted therefrom to the remote compute and store server 106) and/or automatically via a remote input device (e.g., one of the sensors 112 of the agricultural farm 110 of FIG. 1).

If updated agricultural product data has been received in block 702, the method 700 advances to block 704, in which the remote compute and store server 106 updates agricultural product data of the agricultural product for which the agricultural product data has been received. For example, in some embodiments, in block 706, the remote compute and store server 106 may receive registration details corresponding to a crop (e.g., a variant of corn, soybean, etc.). In another example, in some embodiments, in block 708, the remote compute and store server 106 may receive registration details corresponding to livestock (e.g., a horse, a pig, a cow, etc.).

In block 710, the remote compute and store server 106 performs an analysis of the agricultural product based on the received updated agricultural product data. It should be appreciated that, in some embodiments, the remote compute and store server 106 may employ a machine learning algorithm to perform the analysis. Additionally, in some embodiments, the remote compute and store server 106 may use hysteresis to predict outcomes that may be presented as feedback to a user (e.g., at a computing device 102 on which the user is logged into their account).

In block 712, the remote compute and store server 106 presents feedback to the user based on the analysis performed in block 710 (i.e., the updated analysis). For example, in block 714, in some embodiments, the remote compute and store server 106 may present the feedback by transmitting data to a computing device (e.g., the computing device 102) for display on the computing device. It should be appreciated that, in some embodiments, such as in those embodiments in which the feedback is being displayed by a thick client, the data usable to generate the visual representation may be transmitted to the computing device on which the visual representation is to be prepared, rendered, and displayed. It should be further appreciated that, in other embodiments, such as those embodiments in which the feedback is being displayed by a thin client accessed by the computing device (e.g., via a web server on the remote compute and store server 106), the visual representation may be prepared by the remote compute and store server 106 and the data related thereto may be transmitted to the computing device for rendering and display.

Additionally, in some embodiments, in block 716, the remote compute and store server 106 may initiate an automated function in response to the analysis. It should be appreciated that, in some embodiments, the automated function may be performed in response to a trigger, a setting, or an instruction implemented by a user of a computing device on which the feedback is displayed (e.g., the computing device on which the agriculture management and analysis system 100 is managed). For example, in an illustrative embodiment, the remote compute and store server 106 may transmit a command to one or more actuators 114 of the agricultural farm 110 to perform a particular action. In another illustrative embodiment, the remote compute and store server 106 may transmit a notification (e.g., a text message, an email, a fax, an in-app notification, etc.) to the user that includes the feedback information and/or a hyperlink directed thereto.

It should be appreciated that at least a portion of the methods 600 and 700 may be performed by the agriculture analysis and feedback engine 108 of the remote compute and store server 106 and/or the computing device 102. It should be further appreciated that, in some embodiments, the methods 600 and 700 may be embodied as various instructions stored on a computer-readable media, which may be executed by a processor (e.g., the processor 202 of the computing device 102, the processor 302 of the remote compute and store server 106, etc.), communication circuitry (e.g., the communication circuitry 210 of the computing device 102, the communication circuitry 310 of the remote compute and store server 106), and/or other components of the remote compute and store server 106 and/or the computing device 102 to cause the performance at least a portion of the methods 600 and 700.

The computer-readable media may be embodied as any type of media capable of being read by the agriculture analysis and feedback engine 108 of the remote compute and store server 106 and/or the computing device 102 including, but not limited to, a storage medium (e.g., the memory 206 of the computing device 102, the data storage device 208 of the computing device 102, other memory or data storage devices of the computing device 102, the memory 306 of the remote compute and store server 106, the data storage device 308 of the remote compute and store server 106, other memory or data storage devices of the remote compute and store server 106), portable media readable by a peripheral device of the agriculture analysis and feedback engine 108 of the remote compute and store server 106 and/or the computing device 102, and/or other media.

Referring now to FIG. 8, a dashboard view 800 is shown that includes agricultural data relative to livestock analyzed by the remote compute and store server 106 and presented to a user via a graphical user interface (GUI), such as may be displayed by a logged in user on a computing device 102. The illustrative dashboard view 800 includes a navigation interface 802 that includes a menu of options displaying different visual feedback representations of the agricultural data that has been analyzed (e.g., based on selections by the user). The illustrative navigation interface 802 includes an animal management section, a genetic analysis section, a value forecasting section, a breeding suggestions section, a nutritional recommendations section, a lineage tracking section, and a settings section. It should be appreciated that additional and/or alternative sections may be included in the navigation interface 802, in other embodiments.

The illustrative dashboard view 800 additionally includes a feedback selection interface 804 that is configured allow a user to select what agricultural product is being reviewed, as well as what data is to be displayed for that selected agricultural product (i.e., a view mode). Further, the illustrative dashboard view 800 includes a visualization portion 806 in which the data is displayed in a visualized format. As shown, the data may include the types of agriculture, data associated with the selected type of agriculture, such as traits for a particular agricultural product (e.g., as may be selected by an identifier or tag associated with the particular agricultural product) or a group of agricultural products (e.g., as may be selected by the identifiers or tags associated with the particular agricultural product).

In the illustrative embodiment of FIG. 8, the agricultural product is a herd of cattle and the data includes traits of the herd, including birth weight, maternal calving ease, stayability, heifer pregnancy rate, docility, milk production, residual feed intake, average daily grain, tenderness, USDA marbling score, ribeye area, and fat thickness. It should be appreciated that additional and/or alternative traits may be displayed in other embodiments, such as reserve feed intake, percent choice, and/or any other traits that may be based on the registration details provided to the remote compute and store server 106. In some embodiments, stratification of individual and/or groups of animals for each trait may be indicated by a bar graph, where the bars indicated the average market trait value, and the circles indicate where specific animals or groups of animals fall for that trait. In such embodiments, the larger circles can indicate more animals in that particular group.

It should be appreciated that alternative data may be displayed for other livestock (e.g., hogs) and/or crops. The visualization portion 806 of the dashboard view 800 additionally includes other data, such as relative trait strengths for the selected agricultural product(s) and other metrics of the selected agricultural product(s), such as number of agricultural products, age, weight, gender, and financial data (e.g., a current market value, a forecasted valuation, etc.) associated with the agricultural products. It should be appreciate that other data may be shown in other embodiments and/or on other pages associated with the other sections of the navigation menu 802. For example, the other data may include a peak market time for user to sell cattle, real-time market prices (e.g., as may be indicated for beef prices, pork prices, corn feed prices, etc.).

It should be appreciated that the illustrative embodiments of the platform technology (i.e., the agriculture management and analysis system 100, generally, and the agriculture analysis and feedback engine 108, specifically) of the present disclosure are provided herein by way of examples. While the concepts and technology of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

It will be appreciated that the technology described herein has broad applications. The foregoing embodiments were chosen and described in order to illustrate principles of the technology as well as some practical applications. While certain embodiments have been described and/or exemplified herein, it is contemplated that considerable variation and modification thereof are possible.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment.

Some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

For example, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one of A, B, and C” can mean (A); (B); (C): (A and B); (A and C); (B and C); or (A, B, and C). Similarly, items listed in the form of “at least one of A, B, or C” can mean (A); (B); (C): (A and B); (A and C); (B and C); or (A, B, and C).

The preceding description enables others skilled in the art to utilize the technology in various embodiments and with various modifications as are suited to the particular use contemplated. In accordance with the provisions of the patent statutes, the principles and modes of operation of this disclosure have been explained and illustrated in exemplary embodiments. Accordingly, the present invention is not limited to the particular embodiments described and/or exemplified herein.

It is intended that the scope of disclosure of the present technology be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

The scope of this disclosure should be determined, not only with reference to the above description, but should instead be determined with reference to the appended claims, along with the full scope of equivalents to which such claims are entitled. It is anticipated and intended that future developments will occur in the arts discussed herein, and that the disclosed apparatuses, kits, and methods will be incorporated into such future examples.

Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the disclosure and that the technology within the scope of these claims and their equivalents be covered thereby. In sum, it should be understood that the disclosure is capable of modification and variation and is limited only by the following claims.

1.-34. (canceled)

35. A technology platform for improving product performance comprising:
genomic data of an agricultural product,
nutritional information about a diet of the agricultural product, and
a visual representation of the genomic data, wherein the visual representation is configured to provide breeding, marketing, or sale information regarding the agricultural product to a user.

36. The platform of claim 35, wherein the genetic data comprises at least one of genomic data, proteomic data, metabolomics data, and bioinformatics data, and wherein the bioinformatics data is indicative of genomic data and nutritional data associated with the agricultural product.

37. The platform of claim 35, wherein the genomic data comprises at least one of DNA sequencing data, RNA sequencing data, or gene expression data.

38. The platform of claim 35, wherein the visual representation comprises at least one of a breeding suggestion, a market valuation, a market forecast, and a lineage tracker.

39. The platform of claim 35, wherein the visual representation is configured to be a herd management tool.

40. The platform of claim 39, wherein the agricultural product is one or more animals.

41. The platform of claim 35, wherein the visual representation is configured to enable the user to understand a genetic profile of the agricultural product to determine a breeding, sale, or marketing strategy for the agricultural product.

42. The platform of claim 35, wherein the visual representation is indicative of one or more traits of the agricultural product.

43. The platform of claim 42, wherein the visual representation includes a graphical representation of a bar indicative of the average value for each trait and a graphical representation of a circle indicative of the current value of each trait.

44. The platform of claim 35, wherein the visual representation includes a nutritional recommendation associated with the predetermined nutrient that comprises a fertilizer, water, a feed, or a dietary recommendation.

45. The platform of claim 35, wherein the visual representation comprises a numerical value.

46. The platform of claim 45, wherein the numerical value is configured to represent a breeding strategy, a marketing strategy, or a sale strategy.

47. The platform of claim 45, wherein the one or more animals is a cow or cattle.

48. The platform of claim 45, wherein the numerical value is comprised in a sale of the agricultural product.

49. The platform of claim 35, wherein the visual representation is coupled with real-time market data in marketing or a sale of the agricultural product.

50. A visual representation for providing information about an agricultural product, the visual representation comprising a numerical value configured to reflect genomic data and nutritional information about a diet of the agricultural product.

51. The visual representation of claim 50, wherein the numerical value is indicative of a breeding strategy, a marketing strategy, or a sale strategy.

52. The visual representation of claim 50, wherein the visual representation is coupled with real-time market data in a sale of the agricultural product.

53. A method for improved management of an agricultural product in an agricultural farm, the method comprising:
receiving, by a remote compute and store server, registration details from a user,
wherein the registration details define one or more characteristics of the agricultural product;
providing a sample collection kit to the user in response to receiving the registration details,
pairing a genetic sample of the agricultural product with a specific identifier,
wherein the specific identifier comprises a bar code;
directly or indirectly incorporating genetic data from the user into a database of the remote compute and store server, wherein the genetic data is raw or statistically analyzed data generated from the genetic sample of the agricultural product that defines one or more gene markers of the agricultural product,
collecting the genetic sample with the sample collection kit that is associated with the specific identifier;
analyzing, by the remote compute and store server, the genetic data, wherein
analyzing the genetic data comprises classifying the agricultural product for a response to a predetermined nutrient based on the one or more gene markers; and
generating, by the remote compute and store server, a visual representation of the agricultural product based on the analysis of the genetic data, wherein the visual representation is a numerical value configured to represent a breeding strategy, a marketing strategy, or a sale strategy.

54. The method of claim 53, wherein the visual representation comprises at least one of a breeding suggestion, a market valuation, a market forecast, and a lineage tracker.
